(19) United States
(12) Patent Application Publication
(10) Pub. No.: US 2026/0235594 A1
(43) Pub. Date: Aug. 13, 2026

(54) DEPLETION-LIMITED ANALYTE SENSING WITH NON-EQUILIBRIUM BINDING PROBES

(71) Applicant: UNIVERSITY OF CINCINNATI, Cincinnati, OH (US)

(72) Inventor: Jason Heikenfeld, Cincinnati, OH (US)

(21) Appl. No.: 19/150,727

(22) PCT Filed: Jan. 23, 2024
(86) PCT No.: PCT/US2024/012481

§ 371 Date: Jul. 24, 2025

Related U.S. Application Data
(60) Provisional application No. 63/481,254, filed on Jan. 24, 2023.

(51) Int. Cl. G01N 33/543; G01N 21/64; G01N 27/02; G01N 33/548; G01N 33/68; G01N 33/84

(52) U.S. Cl. CPC G01N 33/54306; G01N 21/64; G01N 27/02; G01N 33/54386; G01N 33/548; G01N 33/68; G01N 33/84

(57) ABSTRACT

A method of determining an initial concentration of an analyte in a sample fluid is provided. The method includes providing a plurality of irreversible probes 182b, the plurality of irreversible probes 182b each including at least one binding site configured to bind to an analyte 180. The method further includes introducing a sample fluid 140b to the irreversible probes 182b allowing binding of the analyte 180 to the irreversible probes 182b at the binding site, the analyte 180 binding to the at least one of the plurality of irreversible probes 182b produces a change in a signal having a signal strength. After the signal strength is unchanged for a period of time, the method includes calculating the initial concentration of the analyte in the sample fluid based on the signal strength.

### CROSS-REFERENCE TO RELATED APPLICATION

This application claims priority to, and the benefit of the filing date of, U.S. Patent Application Ser. No. 63/481,254, filed on Jan. 24, 2023, the disclosure of which is incorporated by reference herein in its entirety.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Antigen-based sensing forms the foundation of many types of modern diagnostics, such as enzyme linked immunosorbent assay (ELISA). ELISA is powerful method for detecting and quantifying a specific protein in a complex mixture. Originally described by Engvall and Perlmann (1971), the method enables analysis of protein samples immobilized in microplate wells (e.g., via the use of 96-well plates) using specific antibodies. The technique has revolutionized immunology and is commonly used in medical research laboratories. ELISA also has commercial applications, including the detection of disease markers and allergens in the diagnostic and food industries.

The ELISA method was made possible because of scientific advances in a number of related fields. For example, technology enabling the production of antigen-specific monoclonal antibodies by Kohler and Milstein (1975) led to their use as probes for detecting analytes, which are individual molecules in complex protein mixtures or tissue samples. Initially, detection was achieved by radioimmunoassay using antibodies labeled with radioisotopes, but because of health risks alternatives were sought. Avramais (1966, 1969) and Pierce (1967) developed methods to chemically link antibodies to biological enzymes whose activities produce a measurable signal with solutions containing appropriate substrates. With the development of fluorescence technology, signal generation using fluorophore-labeled antibodies has also become prevalent, especially in multiplex arrays. Although many variants of ELISA have been developed and used in different situations, they and similar assays all depend on the same basic elements:
(1) Coating/Capture: direct or indirect immobilization of antibodies to the surface of polystyrene microplate wells.
(2) Plate Blocking: addition of irrelevant protein or other molecule to cover all unsaturated surface-binding sites of the microplate wells.
(3) Probing/Detection: incubation with the analyte to be measured that affinity-binds to the antigens. In some cases, the binding can be directly measured (e.g. electrical impedance, optical resonance with a waveguide), and in other cases a secondary antibody tag then binds to the analyte as well the second antibody having an enzyme or fluorescent tag.
(4) Signal Measurement: detection of the signal generated.

In a typical assay designed to detect an analyte in a complex protein mixture, the analyte is immobilized either by direct adsorption or via an antibody adsorbed to the wells of a microplate. The plate is blocked and the analyte is probed with a specific detection antibody. The detection antibody may be directly labeled with a signal-generating enzyme or fluorophore or it may be secondarily probed with an enzyme-or fluor-labeled secondary antibody (or avidin-biotin chemistry, see below). For enzymatic detection, the appropriate enzyme substrate is added. The signal observed is proportional to the amount of analyte in the sample. Washing between steps ensures that only specific (high-affinity) binding events are maintained to cause signal at the final step.

The challenge with many of the capture probes used in these types of sensing formats, such as protein probes or antibody probes, is that although they are affinity based, their binding is irreversible. This can require frequent calibration because there is a strong time dependence to the amount of analyte that binds to the surface. It can also increase measurement error because the measurement is made on a signal that is constantly changing versus a signal that is equilibrating.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Various aspects and embodiments of the disclosed invention are directed to depletion-limited analyte sensing with non-equilibrium binding probes. For example, a method of determining an initial concentration of an analyte in a sample fluid is provided (the initial concentration of the analyte in the sample fluid being defined as the concentration of the analyte in the sample fluid prior to the analyte binding to at least one of the plurality of irreversible probes). The method includes providing a plurality of irreversible probes, the plurality of irreversible probes each including at least one binding site configured to bind to the analyte. The method further includes providing the sample fluid including the analyte. The method further includes introducing the sample fluid to the plurality of irreversible probes to cause binding to at least one of the plurality of irreversible probes at the binding site, wherein the analyte binding to the at least one of the plurality of irreversible probes produces a change in a signal having a signal strength. The method further includes depleting the analyte from being unbound to at least one of the plurality of irreversible probes to the extent that the signal strength is unchanged for a period of time. The method further includes calculating the initial concentration of the analyte in the sample fluid based on the signal strength after depleting the analyte from being unbound to at least one of the plurality of irreversible probes to the extent that the signal strength is unchanged for the period of time.

In a related embodiment, the plurality of irreversible probes are coupled to a substrate.

In a related embodiment, fewer analytes are included in the fluid sample than there are in an aggregate of the binding sites.

In a related embodiment, the sample fluid has a total volume of less than or equal to 35 microliters.

In a related embodiment, the irreversible probes are coated onto a plurality of wells.

In a related embodiment, providing the sample fluid including the analyte comprises the sample fluid being provided to a space included in a device, the space being between the plurality of wells and a second material.

In a related embodiment, the method further includes removing the second material from the device after depleting the analyte from being unbound to at least one of the plurality of irreversible probes to the extent that the signal strength is unchanged for a period of time.

In a related embodiment, the plurality of wells comprises a 96 well assay.

In a related embodiment, the plurality of wells comprise a material configured to wick the sample fluid to form a film on the plurality of wells.

In a related embodiment, the material includes a hydrogel or cellulose.

In a related embodiment, the signal is selected from a group consisting of a pH of the sample fluid, an electrical impedance, an optical resonance, a fluorescent tag, and combinations thereof.

Furthermore, a sensing layer included in a sensing device is provided. The sensing layer includes a plurality of irreversible probes, the irreversible probes are configured to bind to an analyte included in a sample fluid, and the binding of each irreversible probe to each analyte is configured to change a signal strength of a signal. The sensing layer further includes a sample space configured to house the sample fluid, wherein the sample space is limited in volume, producing a depletion-limited analyte sensing scheme.

In a related embodiment, the plurality of irreversible probes are coupled to a substrate.

In a related embodiment, the sample space has a volume of less than or equal to 35 microliters.

In a related embodiment, the plurality of irreversible probes are coated onto a plurality of wells.

In a related embodiment, the plurality of wells comprise a 96 well assay.

In a related embodiment, the plurality of wells comprise a material configured to wick the sample fluid to form a film on the plurality of wells.

In a related embodiment, the material comprises a hydrogel or cellulose.

In a related embodiment, the irreversible probes are included in a channel, the channel having a plurality of sections, each section having a height, and each height being different than the height of each adjacent section.

In a related embodiment, the irreversible probes are selected from a group consisting of antibodies, aptamers, proteins, and combinations thereof.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1A is an illustration of a graph showing the signal strength from a prior art system vs. time.

FIG. 1B is an illustration of embodiment of a sensing layer of a prior art device.

FIG. 1C is an illustration of a graph showing the signal strength from a system according to the present invention vs. time.

FIG. 1D is an illustration showing an embodiment of a sensing layer of the present invention prior to the binding of an analyte to an irreversible probe.

FIG. 1E is an illustration showing an embodiment of a sensing layer of the present invention after the binding of an analyte to an irreversible probe.

FIG. 2 is an illustration showing an embodiment of the present invention.

FIG. 3 is an illustration showing an embodiment of the present invention.

### DEFINITIONS

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “irreversible probe” means a molecule that captures or binds to an analyte but for which the reverse process of releasing or unbinding with the analyte is slow or impossible. For example, antibodies are well known to have irreversible binding. In addition aptamers, while reversible for small molecule analyte binding, often have irreversible binding with large analytes such as proteins. Even a protein itself can be used as the irreversible probe. Numerous other irreversible probes are possible.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “sample fluid” means any solution or fluid that contains at least one analyte to be measured, for example river water, blood, urine, saliva, food processing liquid etc.

As used herein, the term “depletion-limited analyte sensing”, refers to a sensing scheme where the irreversible probes capture most or all of the analyte in the sample fluid. Simply, the sample fluid is mostly or fully depleted of analyte in the sample fluid.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. For example, optics, electrodes, additional chemicals or action may be required for a full measurement, and are well known by those skilled in the art of sensors or diagnostics.

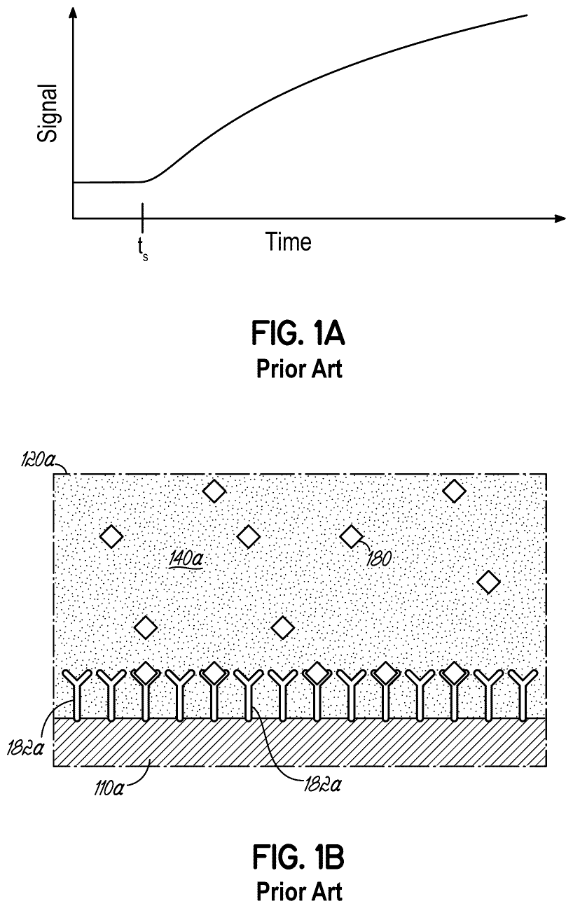

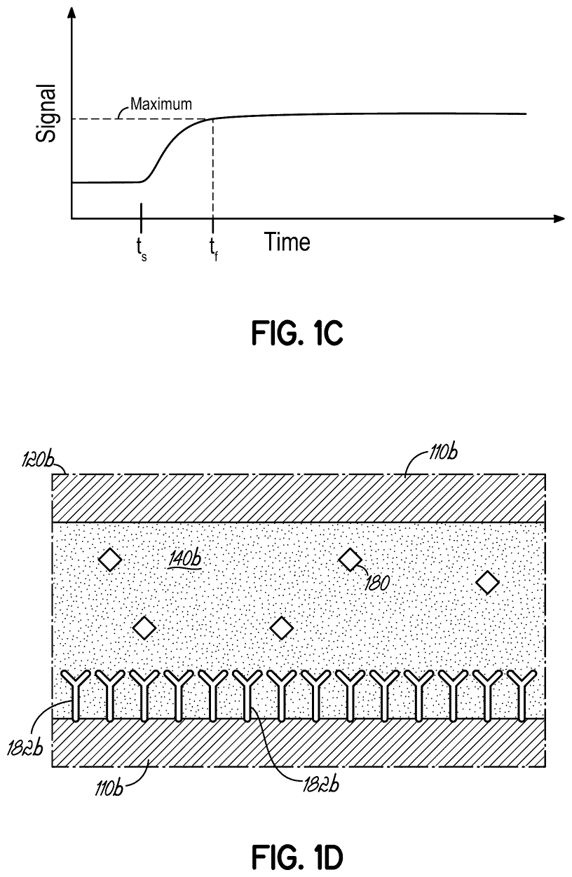

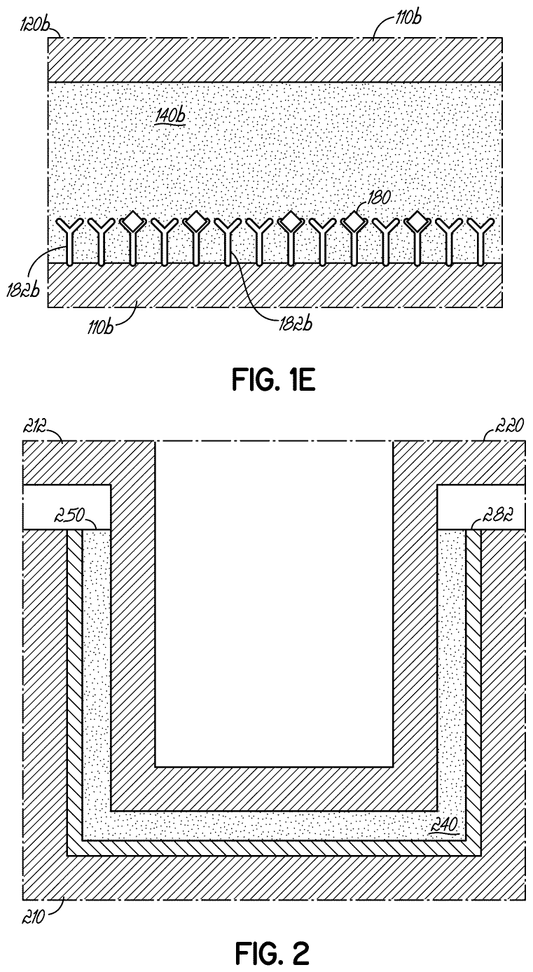

As further background for the present invention, consider a technology where antibodies are placed onto an optical waveguide and as proteins or other analytes are captured by the antibodies the optical signal changes (such as that developed by SiPhox®, headquartered at 111 Terrace Hall Avenue, Burlington, MA). However, the system used by SiPhox® does not reach an equilibrium and the measurement is a transient measurement because the antibodies are irreversible probes. The signal from such a system vs. time could be represented, for example, as shown in FIG. 1A where ‘tₛ’ is the time at which sample is introduced into the system. As shown in FIG. 1B, the sensing layer 120a of such a device has a substrate 110a such as polymer or silicon coupled to irreversible probes 182a. The irreversible probes 182a capture analytes 180 in a sample fluid 140a such as, but not limited to, river water, blood, sweat, urine, saliva, plasma, other biological sample fluid, food processing liquids, or other suitable sample fluids. Because the volume of sample fluid 140a is large, in many assays or detection schemes there is adequate analyte 180 such that irreversible probes 182a will continually capture analyte 180 over time until irreversible probes 182a are fully occupied by analytes 180. Continuous capture of analyte 180 by irreversible probes 182a is a non-equilibrating sensing mechanism which makes it more difficult, time-consuming, and error prone to accurately measure the concentration of analytes 180 in sample fluid 140a compared to other sensing mechanisms, such as those described in embodiments of the present invention.

With reference to FIG. 1D, where like numerals refer to like features, in an embodiment of the disclosed invention, the sample fluid 140b adjacent to the irreversible probes 182b is limited in volume and therefore creates a depletion-limited sensing scheme. FIG. 1D depicts a point in time ts when the sample fluid 140b is introduced to the sensing layer 120b, but prior to the analyte 180 being captured by the irreversible probes 182b. Simply, irreversible probes 182b are able to, and indeed configured to, capture or bind to all or most of the analytes 180 resulting in a more rapid, and/or quantitative, and/or accurate signal for measurement of the concentration of analyte 180 compared to the prior art sensing layer 120a shown in FIG. 1B. For example, as shown in FIG. 1E, the analyte 180 has been completely removed from the sample fluid 140b, and has been irreversibly coupled to irreversible probes 180b.

FIG. 1C represents a signal from the sensing layer 120b shown in FIGS. 1D and 1E vs. time, where ‘tₛ’ is the time at which sample is introduced into the sensing layer 120b (as shown in FIG. 1D), and where ‘t_{f}’ is the time at which most or all analyte 180 in sample fluid 140b has been captured (as shown in FIG. 1E). Particularly, as analyte 180 in the sample fluid 140b becomes bound the irreversible probes 182b a signal strength of a signal changes. When the analyte 180 is sufficiently depleted from the sample fluid 140b to the extent that the signal strength is unchanged for a period of time, that is, time after tr shown in FIG. 1C, it can be concluded that the analyte 180 that was included in the sample fluid 140b has been captured by the irreversible probes 182b, and the concentration of the analyte 180 included in the sample fluid 140b originally fed into the sensing layer 120b can be calculated. This, for example, constitutes a method step of depleting the analyte from being unbound to at least one of the plurality of irreversible probes to the extent that the signal strength is unchanged for a period of time.

With continued reference to FIG. 1E, the sensing layer 120b is shown. In some examples, the sensing layer 120b is included in a device, and is configured to detect and/or calculate based on a measurement, a concentration of the analyte 180 in the sample fluid 140b. The sensing layer 120b is configured to accept a sample fluid 140b. The sample fluid 140b includes the analyte 180 of interest, which is present in the sample fluid 140b in some concentration that it is desired to determine. The sensing layer 120b includes irreversible probes 182b coupled to a substrate 110b. The coupling of the irreversible probes 182b may be mechanical, chemical, or of another nature. The substrate 110b may include any suitable material configured to couple to and/or maintain the irreversible probes 182b rooted in place at least while the sensing layer 120b has accepted the sample fluid 140b, such as polymer or silicon.

The sensing layer 120b further includes irreversible probes 182b. The irreversible probes 182b are molecules that are configured to capture or bind to the analyte 180, but for which the reverse process of releasing or unbinding with the analyte 180 is either slow or impossible. Examples of irreversible probes 182b include antibodies, aptamers, proteins, and combinations thereof. Numerous other varieties of irreversible probes 182b are possible. The irreversible probes 182b are coupled to the substrate 110b, and rooted in place by mechanical, chemical, or other suitable means. Each of the irreversible probes 182b includes a site or a plurality of sites which are configured to accept and capture to the analyte 180, and remove the analyte from freely moving through the sample fluid 140b. The capturing or binding of the analyte 180 to the irreversible probe 182b can produce a signal. The strength of that signal may be related to the total number of captures of individual analytes 180 to irreversible probes 182b in the aggregate. Over time, as more individual analytes 180 capture to irreversible probes 182b, the strength of the signal increases, as shown in FIG. 1C. Furthermore, as shown in FIG. 1E, eventually, all or most of the analyte 180 included in the original sample fluid 140b is bound to the irreversible probes 182b. At this time ‘t_{f}’, a maximum signal strength is reached, and, as shown in FIG. 1C, the signal strength steadies or remains unchanged for time greater than time tf. Furthermore, in the alternative, the capturing of the analyte to the irreversible probe 182b may reduce, inhibit, or otherwise lessen a signal previously observed while still being included as an embodiment of the present invention. In such an embodiment, a minimum signal strength (not shown, but would indeed simply be the opposite of the graph shown in FIG. 1C) would indicate that the analyte 180 has been sufficiently bound to the irreversible probes 182b. In either embodiment, that is maximizing signal strength or minimizing signal strength, there exists a time ‘t_{f}’ in which the signal strength is maximized or minimized, and remains steady or unchanged after time tf. At any time beyond this time tf, the signal strength can be related to concentration by any suitable relationship.

With reference to FIG. 2, where like numerals refer to like features, in an embodiment of the disclosed invention, a sensing layer 220 can include a depletion-limited analyte sensing scheme being applied to conventional assays such as 96-well plates where the wells 210 are coated with capture probes 282 which include chemistry for capturing or binding to analyte included in the sample fluid 240. In some examples, the sample fluid 240 is wicked into and is confined in a space 250 between the wells 210 and a second material 212 such that a depletion-limited sample scheme is created. For example, sample fluid 240 could be wicked into the space 250 between second material 212 and 210 by capillary force, analyte depletion allowed to occur, and then second material 212 removed and the rest of the steps for a conventional 96-well assay could be performed. In such an embodiment, the second material 212 forms a removable insert that forms at least part of a boundary defining the space 250 until the second material 212 is removed. Alternatively, the second material 212 may be non-removeable, and may be permanently fixed feature of the sensing layer 220, and at least partially defining the space 250. This depletion limited approach therefore brings an additional advantage in that the initial volume of sample fluid 240 may be less than by at least one of 3×, 10×, 30×, 100×, 300×, 1000× of the sample volume typically needed for a conventional 96-well plate process (for example, could work with a simple finger prick volume of blood). In this way, in some examples, the total sample 240 volume may be less than or equal to 50 microliters, less than or equal to 35 microliters, less than or equal to 20 microliters, or less than or equal to 10 microliters. The second material 212 may be a polymer or silicon layer configured to prevent the sample 240 from exiting the space 250 between the second material 212 and the wells 210. Alternately, no second material 212 may need be required, as material 210 could be super-hydrophilic and/or textured and/or covered with a dry hydrogel or wicking material such as cellulose, and therefore self-wicking such that a droplet of sample fluid 240 would quickly wick and form a film that enables a depletion-limited sensing scheme.

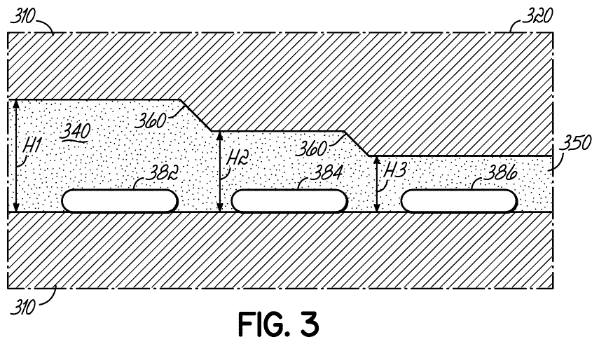

With reference to FIG. 3, where like numerals refer to like features, in an embodiment of the disclosed invention, a sensing layer 320 can include a depletion-limited analyte sensing scheme being applied to other diagnostic tests such as test strips or lateral flow assays. One potential challenge with depletion limited sensing is its dynamic range, which will depend on the volume of the sample fluid 340. Therefore in FIG. 3, the sensing layer 320 includes a channel 350 having a plurality of sections. Each of the plurality of sections in the channel 350 has a height H1, H2, H3 different from adjacent sections. As shown in FIG. 3, a first section of the channel 350 includes a first set of irreversible probes 382. The first section of the channel 350 has a height H1, shown in FIG. 3 as the largest height H1, H2, H3 of all of the sections included in the channel 350. Between each pair of sections of the channel 350 may be a height-reduction portion 360. The height-reduction portion 360 may be a sudden drop in height of the channel 350 (i.e. have a length of 0 m). Indeed, any configuration of height change in the channel 350 is possible in successive sections in order to be included in the invention. In some embodiments, the height-reduction portion 360 may be tapered such as an inclined or declined portion of the substrate 310 in which the height of the channel 350 is either reduced or expanded. While three sections, each having a different height H1, H2, H3 are shown in FIG. 3, in practice, there may be any number of sections in the channel 350. Furthermore, while the first section height H1 is shown in FIG. 3 to be greater than the second section height H2 which is in turn shown as being greater than the third section height H3, in practice, the heights of each section in the channel 350 may be the in any relation in magnitude to each other, so long as they are different in magnitude from the height of adjacent sections. As an example, and as shown in FIG. 3, the individual section heights H1, H2, H3 of irreversible probes for sensing 382, 384, 386 are placed between substrates 310 such that the volume above probes 386 is the smallest and therefore would have the smallest limit of detection in such a scheme, whereas probes 382 would have the highest limit of detection in such a scheme.

The following examples describe an embodiment of the disclosed invention in greater detail.

### EXAMPLES

### Example 1

A diagnostic test or analyte sensing device has an irreversible probe density of 1E11/cm² antibodies and an adjacent volume of sample that is 100 μm thick. 100 μm/cm² is equivalent to 100E-4 cm*1 cm² or 10 μL/cm² . The amount of analyte that can be in the sample (in moles/liter) if the antibodies were to be able to fully deplete the sample would therefore be 1E11 antibodies*1 mole/6.02E23 antibodies=0.166 picomoles. 0.116E-12 moles/10E-6 liters=16 nM. Therefore, this diagnostic test or sensor would be suitable for measuring the low end of free cortisol levels in serum, saliva, or sweat (5-15 nM typically). Cortisol levels can be much higher however in some individuals, and to enable a higher measurement range a second device or the same device can have a second measurement region or area with a thicker layer of fluid, for example 300 μm thick, enabling accurate measurement of up to 48 nM measurement of cortisol, or a third region can be 1000 μm thick enabling measurement up to 160 nM of cortisol.

### Example 2

A B-type natriuretic peptide analyte is provided which is 100 pg/mL*1000 ml/L/(3464 grams/mole) which is ~30 pM. A sensor has a probe density of 1E10/cm² and 10 μm of sample fluid above the probes. Using similar calculations the amount of peptide (analyte) in the sample is ~16 pM. BNP levels can be as high as 300 pM or more, so a second region could also have a sample with thickness 10 μm above the probes, but have a probe density of 1E11/cm² to allow a higher range of detection. Therefore, in addition to sample volume being adjusted for proper detection range, probe density and/or concentration can be adjusted as well.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method of determining an initial concentration of an analyte in a sample fluid, the method comprising:
providing a plurality of irreversible probes, the plurality of irreversible probes each including at least one binding site configured to bind to an analyte;
providing a sample fluid including the analyte;
introducing the sample fluid to the plurality of irreversible probes to allow binding of the analyte to at least one of the plurality of irreversible probes at the binding site, wherein the analyte binding to the at least one of the plurality of irreversible probes produces a change in a signal having a signal strength;
depleting the analyte from being unbound to at least one of the plurality of irreversible probes to the extent that the signal strength is unchanged for a period of time; and
calculating the initial concentration of the analyte in the sample fluid based on the signal strength after depleting the analyte from being unbound to at least one of the plurality of irreversible probes to the extent that the signal strength is unchanged for the period of time.

2. The method of claim 1, wherein the plurality of irreversible probes are coupled to a substrate.

3. The method of claim 1, wherein fewer analytes are included in the fluid sample than there are in an aggregate of the binding sites.

4. The method of claim 1, wherein the sample fluid has a total volume of less than or equal to 35 microliters.

5. The method of claim 1, wherein the irreversible probes are coated onto a plurality of wells.

6. The method of claim 5, wherein providing the sample fluid including the analyte comprises the sample fluid being provided to a space included in a device, the space being between the plurality of wells and a second material.

7. The method of claim 6, further comprising removing the second material from the device after depleting the analyte from being unbound to at least one of the plurality of irreversible probes to the extent that the signal strength is unchanged for a period of time.

8. The method of claim 6, wherein the plurality of wells comprises a 96 well assay.

9. The method of claim 5, wherein the plurality of wells comprise a material configured to wick the sample fluid to form a film on the plurality of wells.

10. The method of claim 9, wherein the material comprises a hydrogel or cellulose.

11. The method of claim 1, wherein the signal is selected from a group consisting of a pH of the sample fluid, an electrical impedance, an optical resonance, a fluorescent tag, and combinations thereof.

12. A sensing layer included in a sensing device, the sensing layer comprising:
a plurality of irreversible probes, the irreversible probes configured to bind to an analyte included in a sample fluid, the binding of each irreversible probe to each analyte configured to change a signal strength of a signal; and
a sample space configured to house the sample fluid, wherein the sample space is limited in volume, producing a depletion-limited analyte sensing scheme.

13. The sensing layer of claim 12, wherein the plurality of irreversible probes are coupled to a substrate.

14. The sensing layer of claim 12, wherein the sample space has a volume of less than or equal to 35 microliters.

15. The sensing layer of claim 12, wherein the plurality of irreversible probes are coated onto a plurality of wells.

16. The sensing layer of claim 15, wherein the plurality of wells comprise a 96 well assay.

17. The sensing layer of claim 15, wherein the plurality of wells comprise a material configured to wick the sample fluid to form a film on the plurality of wells.

18. The sensing layer of claim 17, wherein the material comprises a hydrogel or cellulose.

19. The sensing layer of claim 12, wherein the irreversible probes are included in a channel, the channel having a plurality of sections, each section having a height, and each height being different than the height of each adjacent section.

20. The sensing layer of claim 12, wherein the irreversible probes are selected from a group consisting of antibodies, aptamers, proteins, and combinations thereof.
